(19) United States
(12) Patent Application Publication
(10) Pub. No.: US 2004/0082891 A1
(43) Pub. Date: Apr. 29, 2004

(54) Vacuum splint device

(76) Inventors: David A. Daugherty, Zion, IL (US); Richard C. Liesmann, Antioch, IL (US)

Correspondence Address: Max Shaftal, Patzik, Frank & Samotny Ltd., Suite 900, 150 South Wacker Drive, Chicago, IL, 60606, US

(21) Appl. No.: 10/676,993

(22) Filed: Sep. 30, 2003

Related U.S. Application Data
(60) Provisional application No. 60/415,270, filed on Oct. 1, 2002.

(51) Int. Cl. A61F 5/00

(52) U.S. Cl. 602/5

(57) ABSTRACT

A vacuum splint device for securely immobilizing an injured limb or other body part. The device includes a plurality of T-shaped straps for insertion through slots on the sleeve of the device. Each strap may be folded over the slots and back upon itself to fasten the end of the strap to the device. The sleeve of the device includes a plurality of particles that are initially separated by air to allow for the device to be flexible. Once placed on the injured body part, the air may be removed from the device using an intake/exhaust valve tube assembly to compress the particles together to form a substantially rigid sleeve. A filter on the valve tube assembly ensures that the particles remain within the sleeve. Once the air is removed from the device, a clamp may be used on the tube of the valve tube assembly to prevent any air from flowing into the tube. Upon removal of the device from the injured limb, the straps may be removed from the device for replacement or washing. Air may be introduced into the sleeve by undoing the clamp so as to allow air to reenter the interior and separate the particles and allow the device to be folded.

This application claims priority to U.S. Provisional Patent Application Serial No. 60/415,270, filed Oct. 1, 2002.

### FIELD OF THE INVENTION

This invention relates in general to medical devices and emergency field medical devices, and more particularly to a vacuum splint device for rapid, secure and efficient immobilization of an injured limb or body part, and still more particularly to a vacuum splint device having a plurality of straps having ends for securely attaching the device to the injured limb or body part and retaining the strap on the device.

### BACKGROUND OF THE INVENTION

When limbs or other body parts sustain injuries such as broken bones or ligament damage, it is important to immovably secure the body part as quickly as possible to prevent any further damage to the body part. One way to secure the body part is through the use of an inflatable or vacuum splint device. The device is placed over and/or around the injured body part and secured in place through various fastening means. Once in place, air may be removed from the device to create a vacuum and substantially conform the splint device to the shape of the limb—so as to provide a substantially rigid splint.

Many types of fasteners have been used to try and secure the splint device to the injured limb. One type of fastener used is a conventional belt having a buckle. However, during use, the belts often would slip from side to side and loosen, thereby potentially causing movement of the limb and further damage or injury. Additionally, the movement and location of the buckles against the limb or body part of the injured party may lead to discomfort by the injured party. It was also known to use Velcro® straps that were permanently fastened at one end to the device. However, the Velcro fasteners could not be removed to replace the straps or to clean the straps to remove contaminants and impurities such as dirt or blood which tends to collect thereon.

Other known fastening means utilized multiple additional parts to secure a series of straps to the device for attaching the device around an injured limb. While these known fastening means may work to a degree, use of the additional parts increased the cost to manufacture the device and created a greater likelihood of required maintenance or repair.

It was also known to utilize a plurality of particles inside the sleeve of the device that may be pressed together upon evacuation of the air so as to allow the sleeve to become substantially rigid. However, the particles used were often smaller than the intake/exhaust valve used, thereby allowing some or all of the particles to escape from inside the sleeve.

Therefore, there is a need to produce a vacuum splint device that includes straps that easily, quickly, comfortably and securely fasten the device around an injured limb, while being economical and easy to manufacture and use. There is also a need to produce a vacuum splint device that includes straps that securely fasten the device around an injured limb, while being removable or replaceable for cleaning, replacement, and/or future use. A further need is to produce a vacuum splint device that prevents particles from escaping from the sleeve when the air is evacuated therefrom.

### SUMMARY OF THE INVENTION

The present invention is an improvement over the prior inflatable or vacuum splint devices in that the way that the device is fastened around an injured limb, or other body part, and the way that the particles are retained within the sleeve are unique and improvements over the prior art. In particular, the vacuum splint device of the preferred embodiment of the present invention includes a sleeve, a plurality of substantially T-shaped straps and an intake/exhaust valve tube assembly. After the straps are inserted through slots on the sleeve of the device, the substantially T-shaped ends maintain the straps within the device. The T-shaped end of the strap can use a hook and loop type fastening system or other fastening means, such that the T-shaped end can be folded over onto the strap for retention and to resist undesired loosening. The other end of the strap may also utilize fastening means such as a hook and loop fastening system (e.g., Velcro® to secure the ends of the straps relative to the device.

The sleeve of the vacuum splint device preferably houses a plurality of particles. Air may be introduced into the sleeve to separate the particles or be removed from the sleeve to create a vacuum and allow the particles to compress together to allow the device to become substantially rigid to protect the limb or other body part. The second side of the sleeve is preferably made from a textured or breathable material to provide some degree of air to flow to the injured body part and to help keep the patient's skin from sticking completely to the device. The intake/exhaust valve tube assembly regulates the air flow into and out of the sleeve and includes a filter of screen material to prevent the plurality of particles from exiting the sleeve and a pinch clamp to open or close the air passage through the tube, only as desired.

It is therefore an object of the present invention to provide a new and improved vacuum splint device for attaching to an injured limb or other body part.

A further object of the present invention is to provide a vacuum splint device having a plurality of removable straps for adjustably and securely fastening the device to an injured limb or other body part.

A yet another object of the present invention is to provide a vacuum splint device having a plurality of removable straps that comfortably, efficiently and effectively secure the device over an injured limb or body part.

Another object of the present invention is to provide a vacuum splint device having a mass of movable beads or pellets confined inside a sleeve and a filter associated with the air intake/exhaust valve tube assembly to prohibit the beads or pellets from exiting the sleeve, so as to avoid the need for an inner liner to contain the beads, thereby saving on material and labor expense in manufacturing the device.

A still further object of the present invention is to provide a vacuum splint device made of a textured material that permits air to flow to the patient's skin and tends to resist sticking to the skin of the patient.

A still yet further object of the present invention is to provide a pinch clamp for the intake-exhaust valve tube assembly to control and prevent any air from entering or exiting the sleeve of the device.

A still further object of the present invention is to provide a vacuum splint device that is easy and economical to manufacture.

Other objects, features and advantages of the invention will be apparent from the following detailed disclosure, taken in conjunction with the accompanying sheets of drawings, wherein like reference numerals refer to like parts.

### BRIEF DESCRIPTION OF THE DRAWINGS

FIG. 1 is a top plan view of an embodiment of the vacuum splint device of the present invention showing a plurality of slots for accepting a plurality of T-shaped straps, wherein one of the straps is inserted through a pair of slots and one of the straps is inserted through the straps and folded back upon itself.

FIG. 2 is a bottom plan view of the embodiment of the vacuum splint device shown in FIG. 1.

FIG. 3 is a cross sectional view of the embodiment of the vacuum splint device shown in FIG. 1 illustrating the plurality of particles being separated by air within the sleeve.

FIG. 4 is a top plan view of an embodiment of a T-shaped strap of the present invention.

FIG. 5 is an exploded vertical plan view of the intake/exhaust valve tube assembly of an embodiment of a vacuum splint device illustrating a filter for preventing any filler material from exiting the device and a pinch clamp for regulating the air flow into and out of the device.

FIG. 6 is a vertical plan view of the intake/exhaust valve tube assembly shown in FIG. 5.

FIG. 7 is a perspective view of one embodiment of the pinch clamp for the intake/exhaust valve tube assembly of the present invention.

### DETAILED DESCRIPTION OF THE INVENTION

While this invention is susceptible of embodiment in many different forms, there is shown in the drawings and will herein be described in detail several specific embodiments, with the understanding that the present disclosure is to be considered merely an exemplification of the principles of the invention and the application is limited only to the appended claims.

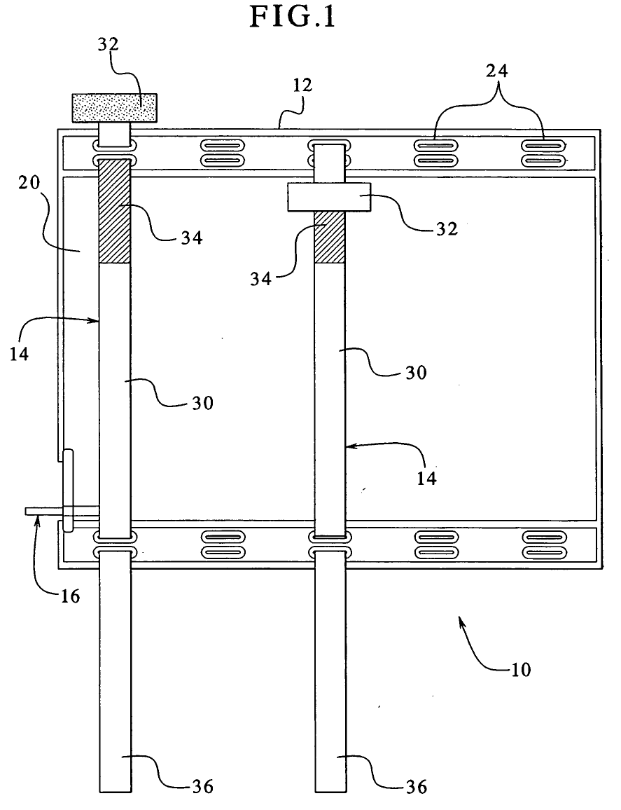

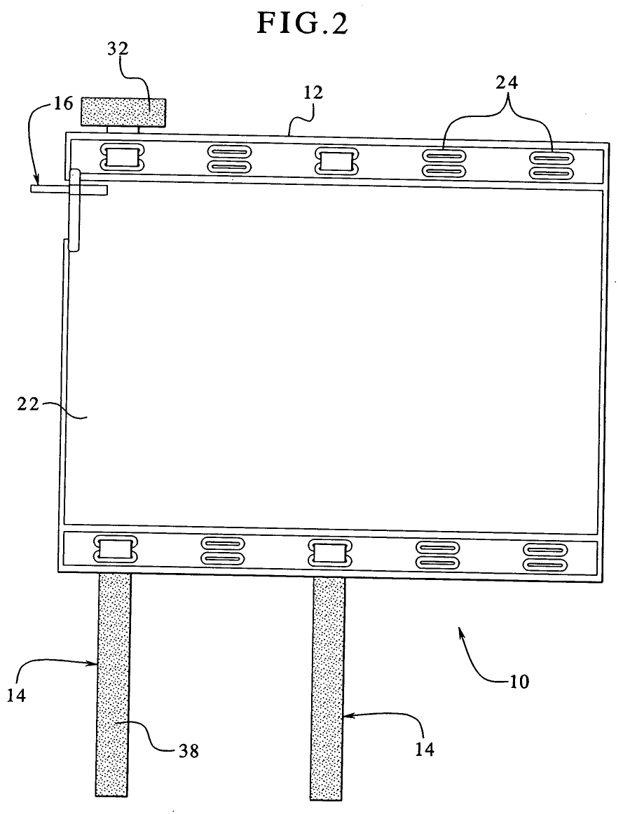

Referring now to the drawings, and particularly to FIGS. 1 and 2, there is shown a preferred embodiment of the present invention. The vacuum splint device, generally designated by the number 10, is shown as having a sleeve 12, a plurality of straps 14 for fastening the device to an injured limb or other body part, and an air intake/exhaust valve tube assembly 16. While a plurality of straps are shown and disclosed, it is appreciated that the present invention may utilize only one strap and not depart from the scope of the present invention.

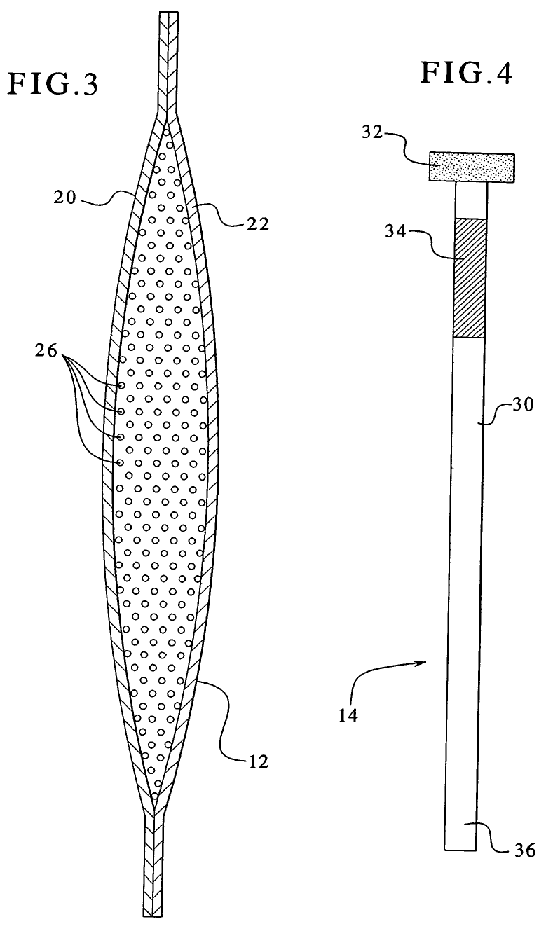

The sleeve 12 preferably includes a first side 20, a second side 22 for contacting an injured limb or other body part, and a plurality of strap holders 24. The first side and second side are attached together in a known way to form the sleeve as shown in FIG. 3 for housing a plurality of particles 26 such as, but not limited to, beads, pellets or sand. It is also appreciated that a separate liner bag or pocket may be located between the first and second sides. While the sleeve is shown as being substantially rectangular in shape, it is appreciated that the sleeve may be sized and shaped to accommodate various body parts and not depart from the scope of the present invention. It is also appreciated that the second side of the sleeve may be made of a textured or breathable material to allow at least some air to flow to the injured body part to resist complete sticking of the material to the body part.

As shown in the figures, the plurality of strap holders are preferably holes or slots 24 extending through the first and second sides 20, 22. The slots or holes 24 of the device are sized to accommodate a plurality of straps 14 for securing the device to an injured body part and are arranged in pairs about the outer periphery of the sleeve. It is appreciated that the holes or slots may be reinforced in a known way to further protect against tearing of the sleeve. While it is preferred that the holes be arranged in pairs about the periphery of the sleeve it is appreciated that they may of any number and located elsewhere of the sleeve. It is further appreciated that the holes may be integral with the surface of the sleeve or they may extend outward in the form of a ring-like device, loop or the like that may be attached to the sleeve by straps, stitching, adhesives or other known means.

The straps include a long strip 30 and an end member 32 on one end of the long strip 30 that has a width that is preferably larger than the width of the slots to prevent the end member from passing through the slots. While the end member may be of any variety of shapes or sizes so long as it is sized to prohibit the end member from passing through the slots, it is preferred that the end member be substantially T-shaped. While an integral strap is shown and disclosed, it is appreciated that the strap may be made from two or more parts. Further, it is appreciated that the retaining end may comprise other objects such as, but not limited to, a bar having a length this is greater than the width of the holes, or a clip or male/female snaps capable of resulting in detachable mounting of the end. It is further appreciated that the ends may have a width that is less than the width of the slot provided that the end is removably and securably attachable about the slot.

To fasten the end of the strap 14 in place, the substantially T-shaped end member 32 may be folded back over and attached to the long strip 30. While the T-shaped end member may be attached to the long strip using any known means including, but not limited to, snaps, clips, rivets, hooks and corresponding holes, zippers, or buttons, it is preferred that the T-shaped end member be attached to the long strip using a hook and loop type fastener such as Velcro®. In particular, the T-shaped end member 32 may contain loops for attaching to hooks 34 located on the long strip 30 of the strap 14, or vice-versa. It is also appreciated that the end may be removably attached to the sleeve and not depart from the scope of the present invention.

The distal end 36 of the straps includes means to fasten the end 36 to the device 10 or strap 14 to securely attach the device 10 to the body part. While it is appreciated that any of a variety of fastening means may be used to fasten the distal end of the straps such as, but not limited to, snaps, clips, rivets, hooks and corresponding holes, zippers or buttons, the distal end of the straps is preferably connected to the long strip of the strap using a hook and loop type fastener such as Velcro. As shown in FIG. 2, the underside of the strap 14 may include loops 38 for attaching to hooks 34 located on the long strip 30 of the strap.

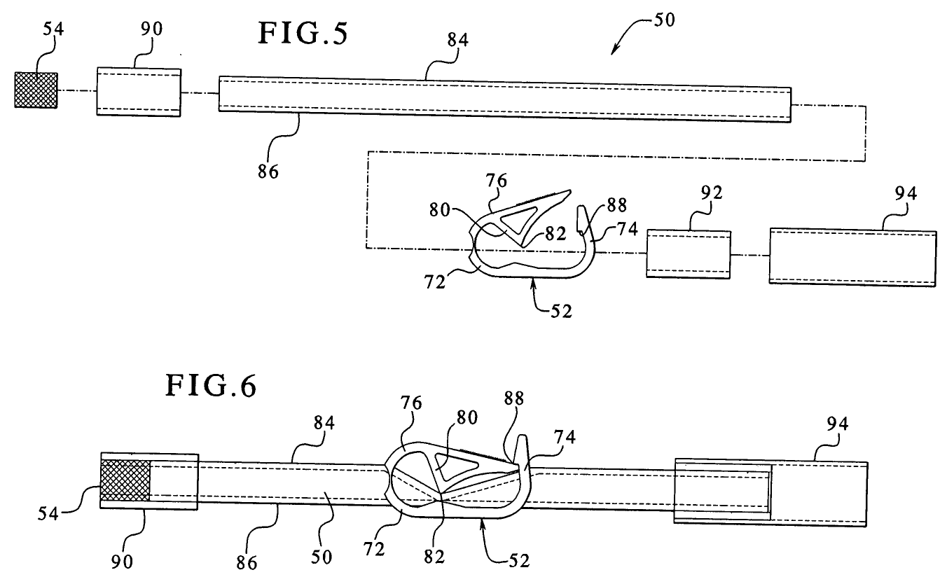

Referring now to FIGS. 5 and 6, an embodiment of the intake/exhaust valve tube assembly is shown. The tube assembly 16, which regulates the amount of air contained within the sleeve 12, includes an elongated tube member 50, a pinch clamp 52 and a filter 54. The tube 50 is preferably made of a material that is substantially rigid to maintain its shape and form, but pliant so as to enable the clamp 52 to bend or deform the tube walls to inhibit air flow. Suitable material may include, but is not limited to, PVC.

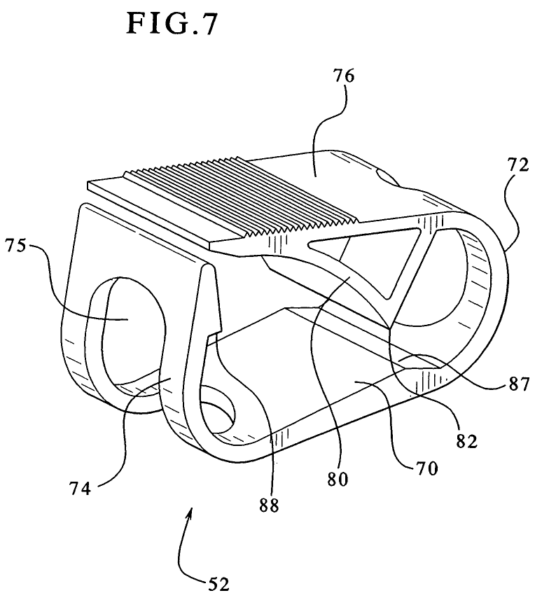

While the clamp may be of any known variety of clamps that can deform the tube walls to inhibit air from flowing through the tube, it is preferred that the clamp be of the form shown in FIGS. 5 through 7. In order to allow the parts of the clip to be resiliently deformed or bent, the clamp is preferably made of a pliant material such as, but not limited to, plastic or metal.

Referring now to FIGS. 5 through 7, the clamp 52 includes a base 70 for accommodating the bottom of the tube, a proximal end 72, a distal end 74 and a top 76. The proximal and distal end members 72, 74 include a hole 75 for accepting the tube. The end members 72, 74, base 70, and top 76 thereby form a passage to allow the tube 50 to be inserted through the clamp 52.

The underside of the top 76 preferably includes a wedge-shaped node 80 that includes a substantially pointed end 82 for pressing the top wall 84 of the tube 50 against the bottom wall 86 of the tube 50 to prevent air from flowing into or out of the sleeve 12. While the node 80 preferably includes a substantially pointed end 82, it is appreciated that the end may be rounded, flat or otherwise and not depart from the scope of the present invention. It is further appreciated that the base 70 of the clamp 52 may include a raised portion 87 that aligns with the pointed end 82 of the node 80 when the clamp is engaged to facilitate the proper clamping of the tube to prohibit air from flowing therein. In order to maintain the clamp engaged against the top wall of the tube, the distal end member 72 of the clamp 52 preferably includes an abutment 88 for retaining the top 76 of the clamp 52 in place. While an abutment is shown, it is appreciated that the top of the clamp may be secured in place through any known means including, but not limited to, notches on the distal end member.

As shown in FIGS. 5 and 6, the filter 54 is placed on one end of the tube 50. The filter may include any of the known filters and be placed inside of the tube or attached to the outside of the tube, but it is preferably a mesh or fine screen material sheet that may be secured over the end of the tube. The mesh sheet 54 may also be comprised of a fabric material that allows air to pass through it in either direction, but will retain the particles inside the sleeve. The mesh sheet 54 may be attached to the end of the tube 50 in any known means, including but not limited to adhesives. Additionally, or in the alternative, the mesh sheet 54 may be secured in place by a bushing 90 that is attached over the end of the tube 50.

In the embodiment shown in the figures, the other end of the tube 50 preferably includes a bushing 92 that acts to retain the clamp 52 on the tube 50. An adapter or mouthpiece 94 may be attached over the bushing 92 to permit the sleeve 12 to be manually or mechanically inflated.

In operation, the vacuum splint device 10 is formed to the shape of the injured limb or body part to be supported. The long strips 30 of the straps 14 pass through the respective pairs of slots 24 and are pulled until the wings of the T-shaped ends 32 of the strap abut against the first side 20 of the device 10 around the slots 24. The ends 32 of the T-shaped straps 14 may then be folded back onto the straps 14 and removably attached thereto using hook and loop fasteners 32, 34 or other known means. Once positioned on the injured body part, the straps 14 may circumscribe the injured body part and device 10 to fasten the device 10 over the body part. The straps 14 may then be removably attached to the device 10 or straps 14 using hook and loop fasteners 34, 38 or other known removable fastening means. Thereafter, a vacuum is created by undoing the clamp 52 and using the air intake/exhaust valve tube assembly 16. Creating a vacuum causes the beads or other particles 26 inside the sleeve 12 to be compacted together to form the desired protective and rigid mold for the injured body part. The filter 54 on the valve 16 prevents any of the particles 26 from escaping from the sleeve 12 during the removal of the air to create the vacuum. After the air is evacuated from the sleeve 12, the top 76 of the clamp 52 is depressed thereby causing the wedge-shaped node 80 to press the top wall 84 of the tube 50 into contact with the bottom wall 86 of the tube 50 to prevent air from flowing into the sleeve 12. The straps 14 may then be readjusted if necessary to assure that the device 10 is securely attached to the injured body part.

To remove the device 10 from an injured body part, the distal ends 36 of the straps 14 are unfastened from the device 10 to permit the sleeve 12 to be opened. After the injured limb is removed, the straps 14 of the device 10 may be removed from the device 10 by pulling the straps 14 through their respective slots 24. The straps 14 may thereafter be replaced or washed for further use. Air may be introduced into the sleeve 12 by flexing the distal end member 74 of the clamp 52 to release the top 76 from the abutment 88 and permit the top 76 and wedge-shaped node 80 to move away from contacting the top wall 84 of the tube 50. After introducing air through the tube 50, the clamp 52 may be re-engaged to maintain the air in the sleeve 12. As shown in FIG. 3, with air in the sleeve 12, the particles 26 are separated, thereby permitting the device to be folded and stored for future use. While a device using a plurality of particles is shown and disclosed, it is appreciated that the present invention may be used on splint devices that do not utilize a plurality of particles.

It will be understood that modifications and variations may be effected without departing from the scope of the novel concepts of the present invention, but it is understood that this application is limited only by the scope of the appended claims.

1. A device for supporting an injured limb or body part comprising:
a sleeve having a first side, a second side for contacting the limb or body part, and at least one strap holder;
at least one strap having a body, a first end and a second end, wherein the at least one strap is inserted through the at least one strap holder;
a first retaining means for removably retaining the first end relative to the sleeve; and
a second retaining means for removably securing the second end relative to the sleeve to secure the straps around the sleeve.

2. The device of claim 1 wherein the second side of the sleeve is made of a textured material so as to allow at least some air to reach the surface of the limb.

3. The device of claim 2 wherein the textured material is an embossed material.

4. The device of claim 1 wherein the strap is substantially T-shaped.

5. The device of claim 4 wherein the at least one strap holder comprises a hole having a hole width and the T-shaped strap comprises a body and an end, wherein the body of the T-shaped strap has a width that is less than the hole width and the end has a width that is larger than the hole width so that the strap may be inserted through the hole and be retained in place by the end of the T-shaped strap.

6. The device of claim 5 wherein the T-shaped strap may be folded over and removably attached to the body of the strap.

7. The device of claim 1 which further includes means to inflate or exhaust air from the device.

8. The device of claim 7 wherein the sleeve further comprises a plurality of particles.

9. The device of claim 8 wherein the sleeve further comprises a bag for retaining the plurality of particles.

10. The device of claim 8 which further includes means to maintain the particles within the sleeve.

11. The device of claim 9 wherein the means to maintain the particles within the sleeve comprises a filter associated with the means to inflate or exhaust air.

12. The device of claim 7 wherein the means to inflate or exhaust air comprises a tube and a clamp for pinching a part of the tube to prohibit air flow.

13. The device of claim 1 wherein the means to secure the straps around the sleeve comprise hook and loop fasteners located on the straps.

14. The device of claim 1 wherein the means to secure the straps around the sleeve comprise snap fasteners.

15. The device of claim 1 wherein the means to secure the straps around the sleeve comprise at least one hook associated with the second retaining means or the strap body and a series of holes associated with the other of the second retaining means or the strap body for mating with the hook.

16. The device of claim 1 which further includes means to secure the retaining end relative to the sleeve.

17. The device of claim 16 wherein the means to secure the retaining end comprises hook and loop fasteners.

18. The device of claim 17 wherein one portion of the hook and loop fasteners is located proximal the second end of the strap and the other portion of the hook and loop fasteners is located on an opposite portion of the strap.

19. The device of claim 16 wherein the means to secure the retaining end comprises snap fasteners.

20. The device of claim 16 wherein the means to secure the retaining end comprises hooks for mating with one of a plurality of holes.

21. The device of claim 1 wherein the at least one strap is removable.

22. The device of claim 1 wherein the at least one strap holders comprises a plurality of holes.

23. The device of claim 22 wherein the plurality of holes are arranged in pairs about the periphery of the sleeve.

24. A device for supporting an injured limb or body part comprising:
a sleeve for supporting the injured limb or body part, the sleeve having a chamber for retaining a plurality of particles, wherein the sleeve is capable of having substantially all of the air within the sleeve evacuated;
means to regulate air into and out of the sleeve; and
a filter to maintain the plurality of particles inside the sleeve.

25. The device of claim 24 wherein the means to regulate air into and out of the device comprises a tube having one end located inside the sleeve, another end located outside the sleeve, and a clamp for pinching part of the tube to maintain the sleeve in the evacuated condition.

26. The device of claim 25 wherein the filter comprises a mesh sheet attached to the end of the tube located inside the sleeve.

27. The device of claim 26 wherein the tube further comprises a bushing for retaining the mesh sheet over the end of the tube.

28. The device of claim 25 wherein the tube further comprises a bushing for maintaining the clamp on the tube.

29. The device of claim 28 wherein the tube further comprises an adaptor that may be placed over the bushing to permit the sleeve to be manually or mechanically inflated.

30. A device for supporting an injured limb or body part comprising:
a sleeve having a first side, a second side for contacting the limb or body part, and a plurality of holes, each of the plurality of holes having a width; and
at least one strap having a body, a retaining end and means to secure the straps around the sleeve, the body of the strap having a width that is less than the width of the holes and the retaining end having a width that is greater than the width of the holes, wherein each of the at least one strap is inserted through at least one of the plurality of holes and retained by the retaining end.
